(19) United States
(12) Patent Application Publication
(10) Pub. No.: US 2008/0214661 A1
(43) Pub. Date: Sep. 4, 2008

(54) Process To Obtain Dibenzylbutyrolactonic, Tetrahydrofuranic Lignans And Their Synthetic And Semi-Synthetic Derivatives, Their Analgesic And Anti-Inflammatory Activities, Topical And/Or Systemic Formulations Containing Said Lignans And Their Respective Therapeutic Method

(75) Inventors: Marcio Luis Andrade e Silva, Bairro Sao Joaquim Franca (BR); Jairo Kenupp Bastos, Ribeirao Preto (BR); Paulo Marcos Donate, Ribeirao Preto (BR); Sergio de Albuquerque, Ribeirao Preto (BR); Rosangela da Silva, Jaboticabal (BR)

Correspondence Address: VOLPE AND KOENIG, P.C., UNITED PLAZA, SUITE 1600, 30 SOUTH 17TH STREET, PHILADELPHIA, PA, 19103, US

(73) Assignee: FUNDACADO DE AMPARO A PESQUISA DO ESTADO DE SAO PAULO, Sao Pailo (BR)

(21) Appl. No.: 11/912,645

(22) PCT Filed: Apr. 28, 2006
(86) PCT No.: PCT/BR06/00084

§ 371 Date: Oct. 25, 2007

(30) Foreign Application Priority Data
Apr. 28, 2005 (BR) PI 0501542 1

(51) Int. Cl. A61K 31/357; C07D 317/46; A61P 29/00; C07D 307/02

(52) U.S. Cl. 514/464; 549/435; 549/429

(57) ABSTRACT

A process to obtain dibenzylbutyrolactonic lignans from (-)-cubebin, isolated from a Piperaceae, especially Piper cubeba, and from (-)-methylpluviatolide, isolated from a Rutacea, especially Zanthoxylum naranjillo; their synthetic and semi-synthetic derivatives and tetrahydrofuranic lignans, such as galgravin and veragensin, isolated from Nectandra megapotamica, as well as the analgesic and anti-inflammatory activities of said lignans, and the topical and/or systemic formulations. Also presented is a therapeutic method using topic and/or systemic formulations based on said lignans for the treatment of inflammation and/or pain. Further, a process to obtain synthetic and semi-synthetic derivatives of (-)-cubebin, such as: (-)—O-acetyl cubebin; (-)—O-methyl cubebin; (-)—O—(N,N-dimethylamino-ethyl)-cubebin; (-)-hinokinin; (-)-6,6′-dinitroinokinin; (-)—O-benzyl cubebin; (-)-6,6′-diaminohinokinin and other synthetic derivatives which may be obtained, and synthetic and semi-synthetic derivatives of (-)-methylpluviatolide, such as (-)-6,6′-dinitromethylpluviatolide and (-)-6,6′-diaminomethylpluviatolide, to be used in the manufacture of medicine that has analgesic and anti-inflammatory activity is presented.

### FIELD OF INVENTION

The present invention refers to a process to obtain dibenzylbutyrolactonic lignans from (-)-cubebin, isolated from a Piperaceae, especially Piper cubeba, and from (-)-methylpluviatolide, isolated from a Rutacea, especially Zanthoxylum naranjillo; their synthetic and semi-synthetic derivatives and tetrahydrofuranic lignans, such as galgravin and veragensin, isolated from Nectandra megapotamica, as well as the analgesic and anti-inflammatory activities of said lignans, and the topical and/or systemic formulations in which lignans represent 60 to 80% of the formulation. The invention also refers to a therapeutic method using topic and/or systemic formulations based on said lignans for the treatment of inflammation and/or pain.

### BACKGROUND

More specifically, it refers to a process to obtain synthetic and semi-synthetic derivatives of (-)-cubebin, such as: (-)—O-acetyl cubebin; (-)—O-methyl cubebin; (-)—O—(N,N-dimethylamino-ethyl)-cubebin; (-)-hinokinin; (-)-6,6′-dinitroinokinin; (-)—O-benzyl cubebin; (-)-6,6′-diaminoinokinin and other synthetic derivatives which may be obtained, and synthetic and semi-synthetic derivatives of (-)-methylpluviatolide, such as (-)-6,6′-dinitromethylpluviatolide and (-)-6,6′-diaminomethylpluviatolide, to be used in the manufacture of medicine that have analgesic and anti-inflammatory activity. The present invention also refers to the process to obtain the substances galgravin and veragensin isolated from Nectandra megapotamica, as well as their synthetic and semi-synthetic derivatives with substituents on the aromatic rings to be obtained.

The search for new therapeutic alternatives that are safer and more effective is extremely important to overcome current. The Lignins, described here, present excellent analgesic-anti-inflammatory activity and practically no side effects for their use.

With the technological development, deeper studies have shown researchers and the pharmaceutical industry the need to synthesize bioactive substances, having natural products as their raw materials. Many classes of different natural products have been synthetize new pharmaceuticals, such as terpene derivatives used as raw materials for the synthesis of artemisin and sesquiterpene derivative with important anti-malaria activities. The class of lignans, in which (-)-cubebin is included, is of particular interest since, besides the activities already mentioned, they present anti-tumor, anti-viral and anti-Chagas activities. Among the various therapeutic applications of plants, many present anti-inflammatory and analgesic activity, which are widely used in popular medicine. Thus research and studies to confirm said activities, and toxicity profile, in biological assays is needed.

Inflammation process is started and conducted by mediators of cell and plasma origin which, by acting locally, will promote the characteristic signals of said response, i. e. pain, heat, redness and tumor, followed or not by loss of function of the affected organs or tissues. Clinically, the inflammatory reaction appears in a stereotyped manner and independent from the nature of stimulation. Small variations may occur depending on the affected tissue or organ and the coexistence of pathological states.

Arachidonic acid cascade is responsible for the biotransformation of important cell mediators. Among these, we find prostaglandins (PGs), which are highly important in various physiologic processes. Currently, the researches in new non-steroidal anti-inflammatories (AINS) have been made for the selective inhibition of enzymes of the arachidonic acid cascade. Recently, with the discovery of a second isoform of prostaglandin-endoperoxide synthetase (PGHS), PGHS-2, the treatment of inflammatory diseases gained new perspectives with the possibility of disclosure of a new class of AINS agents, which would act without the side effects caused by the classic anti-inflammatories.

Research showed that dibenzylbutyrolactonic lignans such as (-)-cubebin, as well as synthetic and semi-synthetic derivatives (-)—O-acetyl cubebin; (-)—O-methyl cubebin; (-)—O—(N,N-dimethylaminoethyl)-cubebin; (-)—O-benzyl cubebin; (-)-hinokinin; (-)-6,6′-dinitroinokinin; (-)-6,6′-diaminohinokinin and tetrahydrofuranic lignans, such as galgravin and veragensin, isolated from husks of Nectandra megapotamica, have significant analgesic and anti-inflammatory activity.

### SUMMARY

The objective of the invention proposed is to obtain dibenzylbutyrolactonic lignans, such as: (-)—O-acetyl cubebin (2), (-)—O-methyl cubebin (3), (-)—O—(N,N-dimethylaminoethyl)-cubebin (4), (-)—O-benzyl cubebin (5), (-)-hinokinin (6), (-)-6,6′-dinitroinokinin (7), (-)-6,6′-diaminohinokinin (8), derivatives from (-)-cubebin (1), isolated from Piper cubeba, as well as other dibenzylbutyrolactonic lignans derived from methylpluviatolide (9) which is isolated from Rutacea, such as: (-)-6,6′-dinitromethylpluviatolide (10) and (-)-6,6′-diaminomethylpluviatolide (11) besides the tetrahydrofuranic lignans, such as galgravin (12) and veragensin (13), isolated from Nectandra megapotamica and others that may be obtained by the processes as described here; which will be used in the manufacture of medicine that may act as analgesic-anti-inflammatory with almost 100% of power as per the following chemical structures.

Structures of Cubebin (1) and dibenzylbutyrolactonic lignan derivatives:

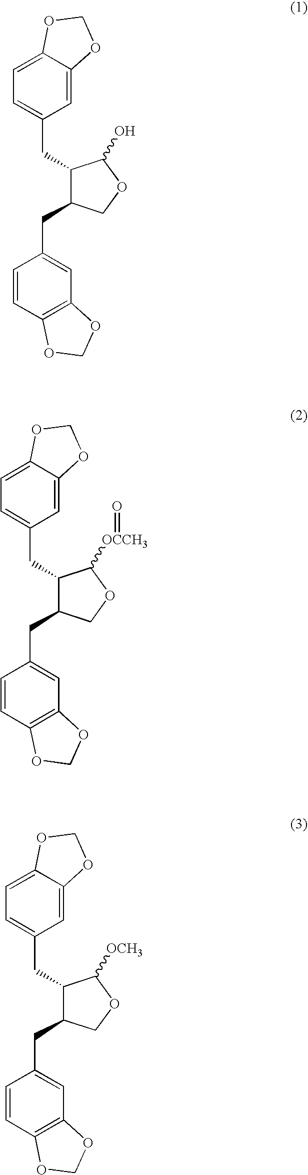

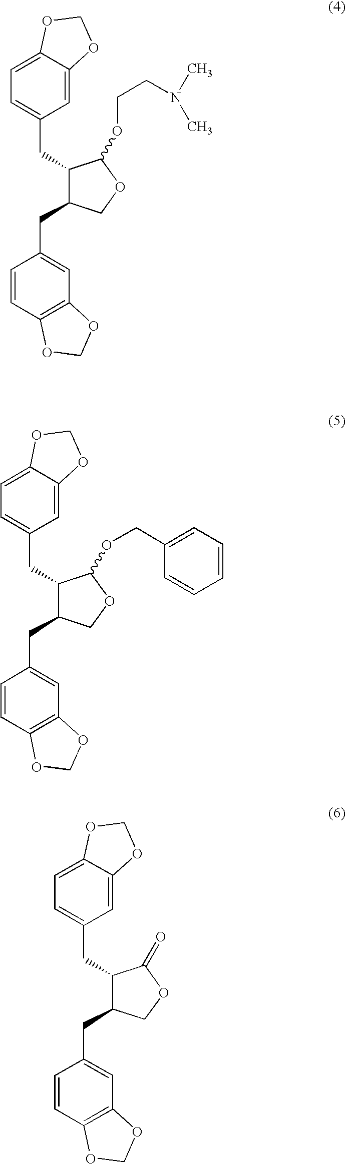

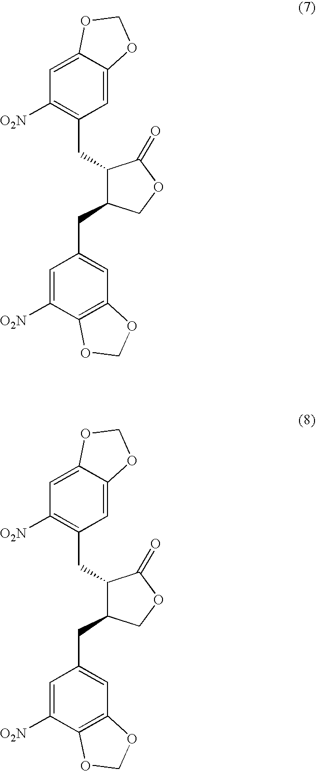

(-)-Methylpluviatolide (9) derivatives, obtained by full synthesis from veratraldehyde and methyl succinate, since they have methoxy groups in one of their aromatic rings, as well as —NO₂ and —NH₂ substituents, present large analgesic-anti-inflammatory potential.

Methylpluviatolide (9) structures and their dibenzylbutyrolactonic lignan derivatives:

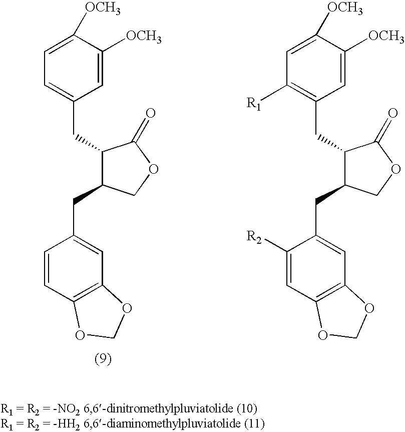

Tetrahydrofuranic lignans, such as galgravin (12) and veragensin (13), have been used as important antagonists with action on receptors involved with PAF (blood platelet aggregation factor). Said lignans present important analgesic and anti-inflammatory activities, such as dibenzylbutyrolactonic lignans isolated from Z. naranjillo and P. cubeba.

Galgravin (12) and veragensin (13) structure:

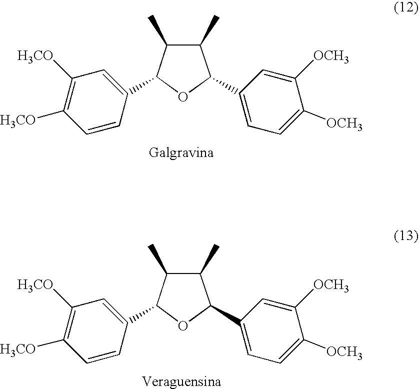

### BRIEF DESCRIPTION OF THE DRAWINGS

To better illustrate and help to understand the invention proposed, the following figures are presented:

FIG. 1—flow diagram of the process to obtain dibenzylbutyrolactonic lignans from Piper cubeba seeds and Zanthoxylum naranjillo leaves;

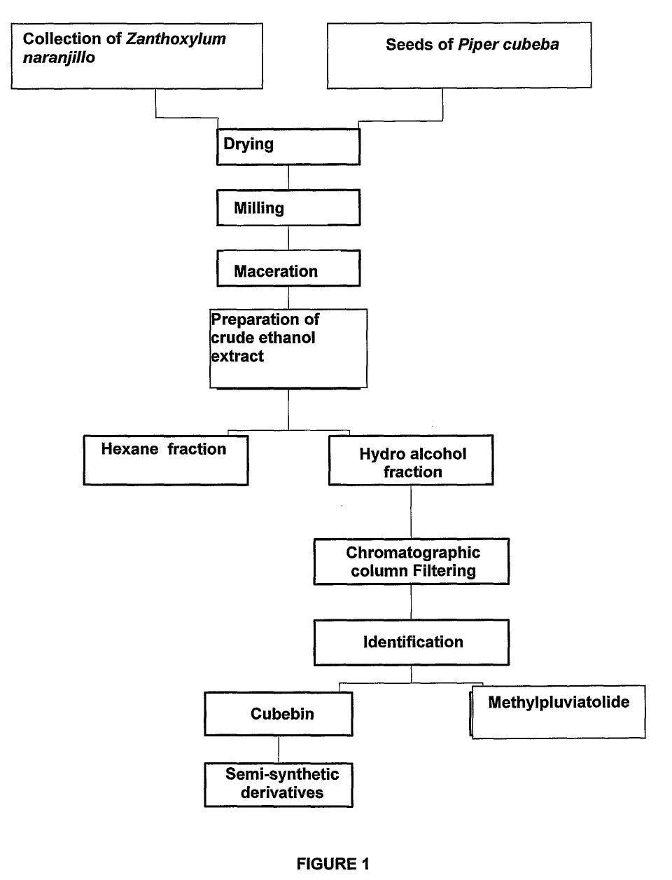

FIG. 2—flow diagram of the process to obtain tetrahydrofuranic lignans from Nectandra megapotamica;

FIG. 3—graphs of the effect of oral administration of (-)-cubebin (1), (-)—O-benzyl cubebin (5), (-)-hinokinin (6), (-)-6,6′-dinitroinokinin (7) and (-)-6,6′-diaminohinokinin (8) in the rat foot edema.

FIG. 4—graphs of the effect of oral administration of (-)-cubebin (1), (-)—O-benzyl cubebin (5), (-)-hinokinin (6), (-)-6,6′-dinitroinokinin (7) and (-)-6,6′-diaminohinokinin (8) in the contortion test in mice.

### DETAILED DESCRIPTION OF THE PREFERRED EMBODIMENTS

The processes to obtain tetrahydrofuranic lignans from Nectandra megapotamica and (-)-cubebin (1) derivatives, dibenzylbutyrolactonic lignan, from Piper cubeba, have the following steps:
Collection;
Drying;
Milling;
Maceration;
Preparation of extract;
Fractioning and filtering;
Purification;
Identification.

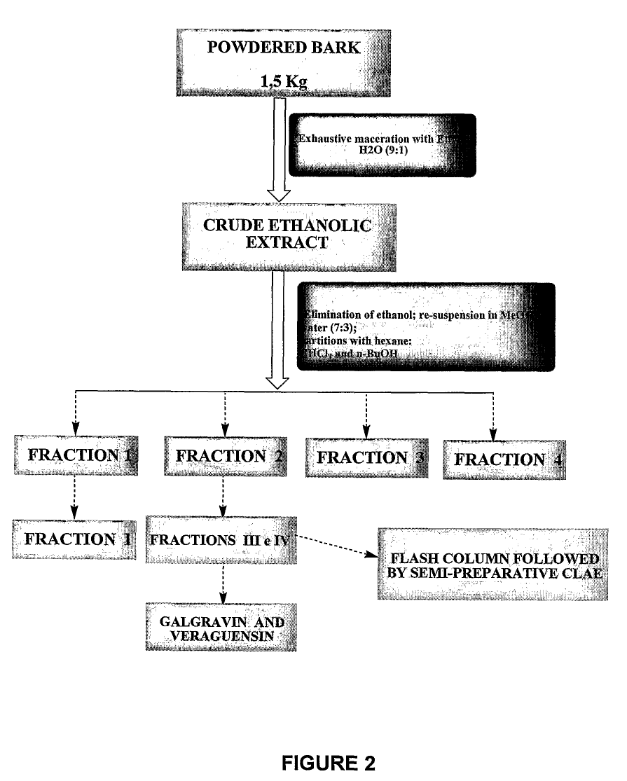

The process to obtain tetrahydrofuranic lignans galgravin (12) and veragensin (13), shown in FIG. 2, comprises the following steps:
a) Collection: husks of Nectandra megapotamica;
b) Drying: oven at temperature between 40-60° C.;
c) Milling: Nectandra megapotamica husks were pulverized in a knife mill;
d) Maceration: the powder of Nectandra megapotamica husks obtained was exhaustively extracted from EtOH: H₂O (9:1) at 25° C. for five days;
e) Preparation of crude extract: the product from maceration was filtered and concentrated under reduced pressure at the temperature of 30° C. until the complete elimination of solvent.
f) Fractioning of extract: the crude extract, obtained from the Nectandra megapotamica husks, was dissolved in MeOH: H₂O (7:3), followed by repeated partitions with hexane, chloroform and butanol. The remaining water fraction was lyophilized. Fraction I, obtained from partition, was chromatographed in silica gel on a liquid chromatography system in vacuum column, using mixture hexane-ethyl acetate in growing proportions, resulting in 4 fractions. The resulting Fraction I (hexane-EtOAc 9:1). Fraction II, obtained from partition, was chromatographed in silica gel on a liquid chromatography system in vacuum column, using mixture hexane-ethyl acetate in growing proportions of, resulting in 6 fractions. The resulting fraction III (hexane-EtOAc 1:1) and fraction IV (hexane-EtOAc 4:6) obtained from fraction II were submitted to flash column chromatography over silica, using hexane-EtOAc (9:1) as a mobile phase followed by semi-preparative HPLC (high performance liquid chromatography) (MeOH—H₂O 75: 250). By this process, the compounds galgravin (12) and veragensin (13) are obtained.
g) Identification: made by the analysis of the data of the nuclear magnetic resonance (NMR) of ¹H and ¹³C, [α]D, Mass, IV.

In Scheme 1, the obtaining reactions are illustrated with the corresponding structures of the semi-synthetic derivatives of (-)-cubebin (1), isolated from Piper cubeba seeds, which consist of the following stages: i; ii; iii; iv, v, vi and vii.

i) (-)-Cubebin (1), a dibenzylbutyrolactonic lignan, had its structured modified by semi-synthesis with the purpose to improve its biological activity. (-)-Cubebin (above 200 g) and P. A. acetic anhydride reacted in pyridine. Cubebin was dissolved in acetic anhydride, and pyridine was added under constant shaking at room temperature during the whole period of reaction (24 h). After the reaction, thin layer chromatography analysis was conducted (silica gel 60—mobile phase: hexane/AcOEt 6:4).

The isolation of (-)—O-acetyl cubebin (2) was made by the addition to the reaction medium of portions of toluene and successive evaporations at reduced pressure to extract pyridine. After this procedure, portions of dichloromethane were added to the medium containing toluene and successive evaporations under reduced pressure to eliminate toluene. The organic phase was then transferred to a collecting flask and purification in preparative circular chromatography followed (CCP) (CROMATOTRON). After this procedure, the product (-)—O-acetyl cubebin (2) was submitted to purity determination in high performance liquid chromatography (HPLC), finding a purity index >95%; The product (-)—O-acetyl cubebin (2) was taken to NMR analysis of ¹H and ¹³C and [α]²⁶_{D}.

ii) To (-)-cubebin (1) (above 200 g) in distilled and dry THF, NaH was added (sufficient quantity, washed with hexane free from paraffin grease), shaking the mixture for % hour at room temperature. Methyl iodide was then added, and the reaction medium was left under shaking during the night atr N₂ atmosphere.

The isolation was made by the decomposition of excess NaH by the addition of methanol in water (1:1). Organic solvents were distilled from the reaction medium. Diluted HCl was added and extracted with AcOEt. The organic phase was neutralized with a 5% NaHCO₃ solution, saline solution (10% NaCl), again with 5% NaHCO₃ solution, dried with MgSO₄ and filtered. The solvent rotaevaporated and a brown residue were obtained. Subsequently, the product was submitted to silica gel column chromatography (eluent hexane/AcOEt 4:1). Purification was made by circular preparative chromatography (CPC) (CROMATOTRON). After this procedure, the product (-)—O-methyl cubebin (3) was submitted to purity determination by HPLC, finding a purity index of 98%. Characterization was made by NMR of ¹H and ¹³C and [α]²⁶_{D}.

iii) 300 g (701.5 mmol) of (-)-cubebin (1) in 5 l of ethanol in a solution of sodium ethoxide (5 l of ethanol, 2 MEq of Na⁰) were added over 2 hours of reflow. Subsequently, 120 g (1020 mmol) of dimethylethylamine chloride were added. The reaction was monitored by CCD and the reflow was prolonged more 4 four hours. At the end of the reaction, 5 ml of water were added, the phases were separated and the organic phase was extracted with ethyl acetate (3×500 ml). The organic phase was washed with a 10% water NaCl solution (3×500 ml), dried with MgSO₄ and filtered. The solvent was evaporated at reduced pressure and the residue was purified over a silica gel chromatography column by using dichloromethane as eluent. The product (-)—O—(N,N-dimethylaminoethyl)-cubebin (4) was obtained as a dark yellow oil and its purity was estimated at 99% by HPLC.

iv) (-)-Cubebin (1) (above 200 g) was reacted with 2 molar equivalents of pcc (piridinium chlorochromate) in dry dichloromethane. In a 3-mouth balloon, pcc (piridinium chlorochromate) was put and dry dichloromethane was quickly added to avoid its decomposition. The system remained under constant shaking and inert atmosphere (N₂). For each gram of pcc, 1 l of dichloromethane was used. In a separate balloon, (-)-cubebin (1) was dissolved in dry DCM, also keeping the inert atmosphere. With a Teflon hypodermic syringe and a wide caliber needle, the solution (suspension) was taken from the balloon and added in drops to the balloon containing pcc, keeping shaking and N₂ atmosphere for 24 hours. After purification, it was submitted NMR spectroscopic analysis of ¹H and ¹³C. The product obtained was as dark yellow oil and its purity was estimated at 99% by HPLC.

After this, the reaction medium was poured into a chromatographic column with sintered plate n^{o} 2 containing mono-hydrated MgSO₄ and vacuum filtered. The sample was then submitted to chromatographic column, using column with a sintered plate n^{o}2, silica gel 60 for the column and the solvent systems: pure hexane, 8:2 hexane/AcOEt, 7:3 hexane/AcOEt, 6:4 hexane/AcOEt, 1:1 hexane/AcOEt and 100% AcOEt. After elution, the solvent was eliminated in a turning evaporator and the resulting product was purified in rotating preparative chromatography, resulting in (-)-hinokinin (6). After purification, it was submitted to NMR spectroscopic analysis of ¹H and ¹³C and [α]²⁶_{D}. The product (-)-hinokinin (6) was obtained as a dark yellow oil and its purity was estimated at 99% by HPLC.

v) (-)-Hinokinin (6) (above 200 g) was dissolved in chloroform, keeping the reaction medium at −6° C. Nitric acid (6 MEq) was slowly added by drops, letting it to react for 2 hours. After this period, a Na₂CO₃ saturated solution was added to end the reaction.

6,6′-Dinitroinokinin (7) was extracted from the reaction medium with chloroform, which was evaporated under reduced pressure. After recrystallization in methanol, a yellow powder product 6,6′-dinitroinokinin (7) was obtained. After purification, it was submitted to NMR spectroscopic analysis of ¹H and ¹³C and [α] ²⁶_{D}. The product 6,6′-dinitroinokinin (7) was obtained as a dark yellow solid and its purity was estimated at 98% by HPLC and other spectral data.

vi) A solution of (-)-cubebin (1) (300 g, 701.5 mmol) in 5 l of THF was added to a suspension of NaH (sufficient quantity washed with hexane free from grease) in THF (3 l), shaking the mixture for 30 minutes at room temperature. Benzyl bromide (250 ml) was then added and the reaction medium was shaken for one night under N₂ atmosphere. Excess NaH was decomposed by the addition of methanol in water (1:1). Diluted HCl was added and the medium was partitioned three times with ethyl acetate (3×500 ml). The organic phase was neutralized with a 5% NaHCO₃ water solution (2×500 ml), 10% NaCl solution in water (3×500 ml) and 5% NaHCO₃ solution in water (2×500 ml), dried with MgSO₄ and filtered. The solvent was evaporated under reduced pressure, obtaining a brown residue which was purified in a silica gel column using hexane/ethyl acetate (4:1) as eluent, providing transparent oil with estoichiometric yield of 91.4%. The product (-)—O-benzyl cubebin (5) had its purity estimated at 98% by HPLC and other spectral data.

vii) To an autoclave of stainless steel, 300 g (687.3 mmol) of the compound 6,6′-dinitroinokinin (7) dissolved in 10 l of anhydrous methanol was added under shaking and then 298.8 g of palladium (5%) over activated charcoal carbon in anhydrous methanol (5 l). The autoclave was closed and submitted to 20 atm of H₂ for 24 hours at room temperature. The suspension was filtered through silica gel and the solvent was evaporated under reduced pressure. The product (-)-6,6′-diaminohinokinin (8) was purified by silica gel column chromatography using as eluent the mixture of hexane-ethyl acetate at 1:1 proportion. The product had its purity estimated at 98% by HPLC and other spectral data.

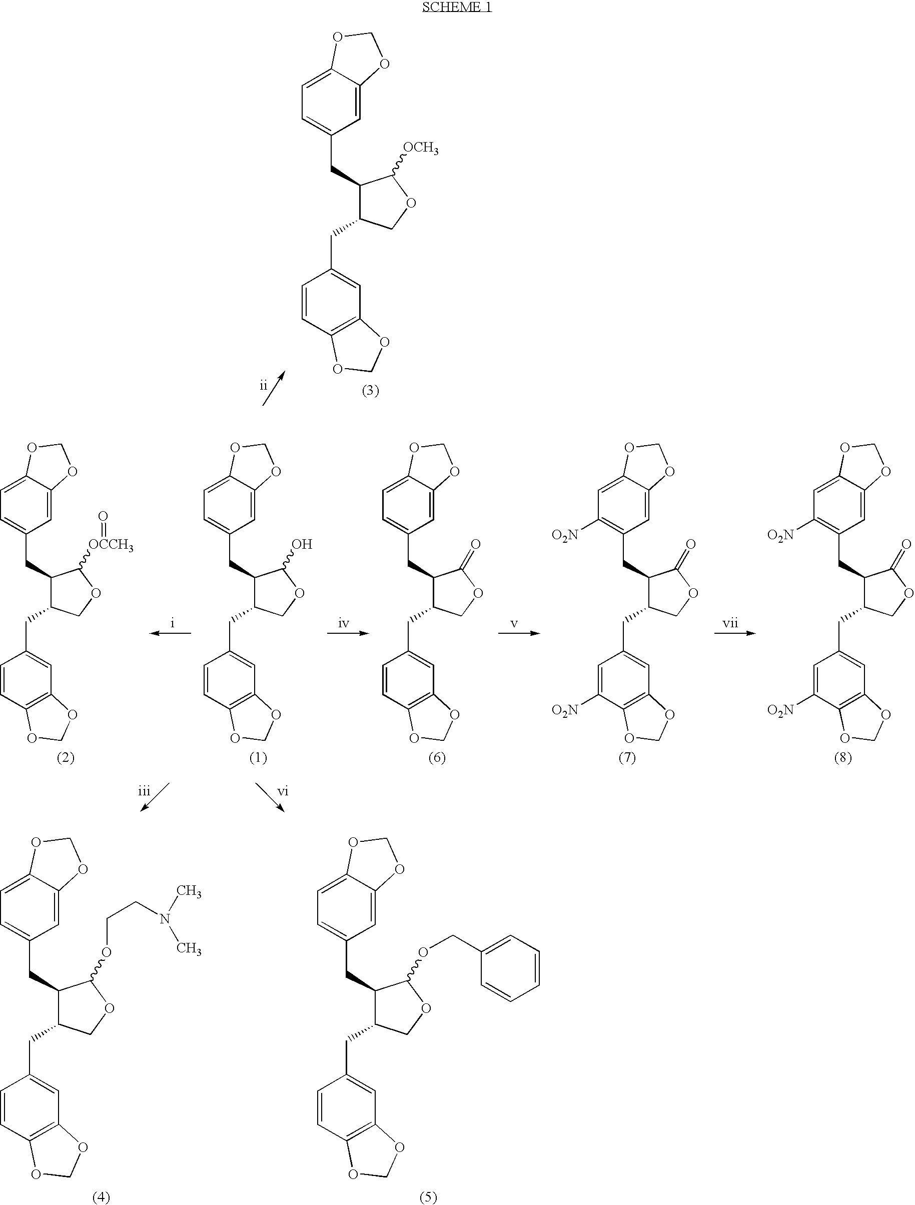

In Scheme 2, obtaining reactions are illustrated with the corresponding structures of the semi-synthetic derivatives of methylpluviatolide (9), isolated from leaves of Zanthoxylum naranjillo, which consist of the following stages: viii, ix, x, xi.

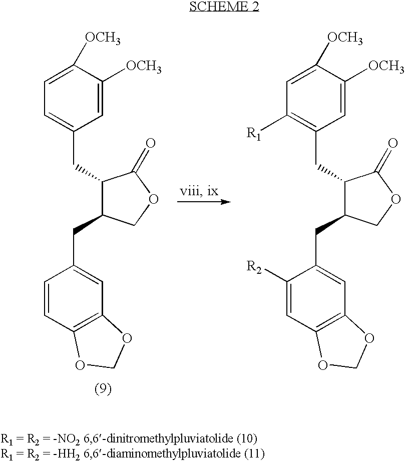

The derivatives (-)-6,6′-dinitromethylpluviatolide (10) and (-)-6,6′-diaminomethylpluviatolide (11) were respectively obtained by means of the following procedures:

viii) same procedure to obtain 6,6′-dinitroinokinin (7), but using methylpluviatolide (9) instead of hinokinin (6), thus obtaining the derivative 6,6′-dinitromethylpluviatolide (10).

ix) same procedure to obtain (-)-6,6′-diaminohinokinin (8), but from 6,6′-dinitromethylpluviatolide (10) and obtaining the derivative 6,6′-diaminomethylpluviatolide (11).

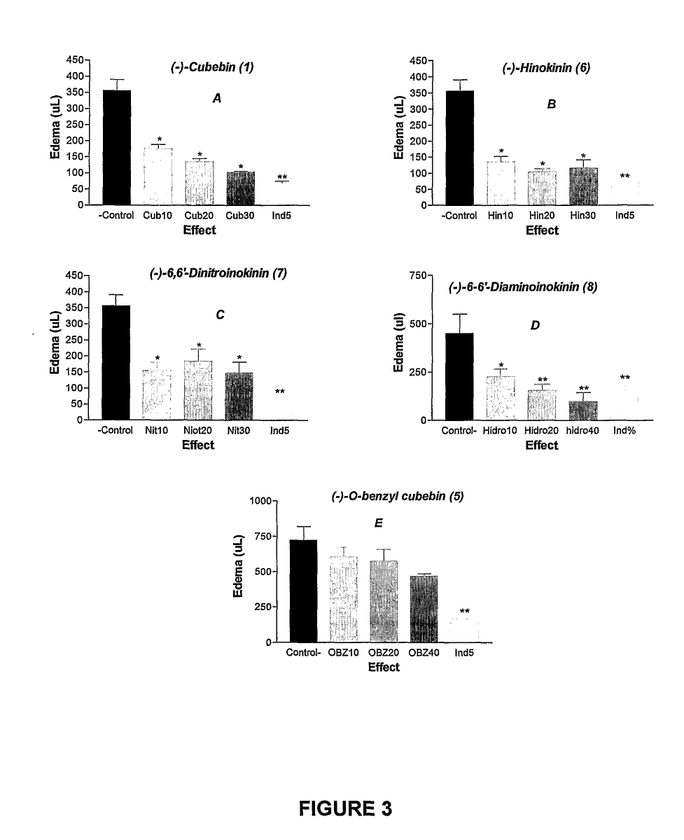

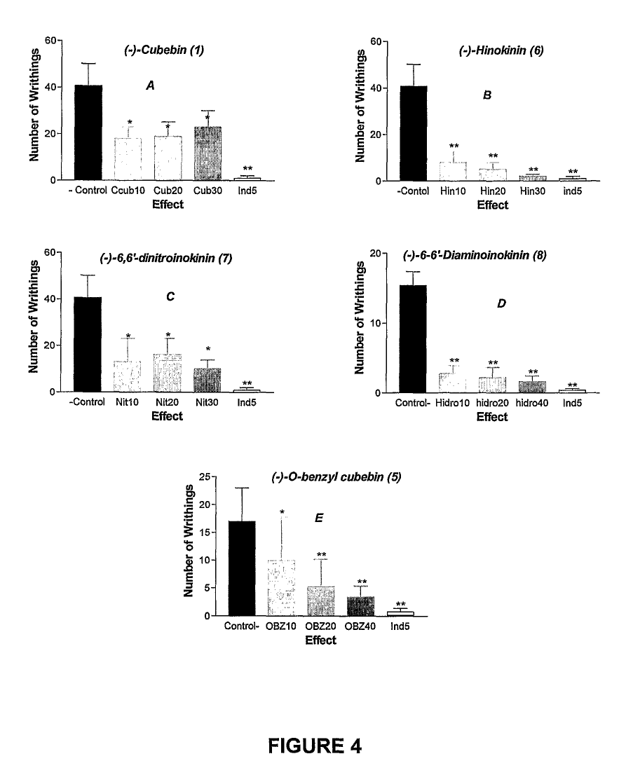

Besides the results related to trypanocidal activity already presented, which have already generated a patent application, analgesic and anti-inflammatory activities of various (-)-cubebin derivatives were analyzed of which (-)-hinokinin (6), (-)-6,6′-dinitroinokinin (7) and (-)-6,6′-diaminohinokinin (8) showed higher efficacy as anti-inflammatory agents, inhibiting 71%, 62% and 82%, respectively (FIG. 3—A, B, C and D). The derivative (-)—O-benzyl cubebin (5) was not efficient as an anti-inflammatory agent. Concerning analgesic activity, derivatives (-)-hinokinin (6), (-)-6,6′-dinitroinokinin (7), (-)-6,6′-diaminohinokinin (8) and (-)—O-benzyl cubebin (5) were efficient as analgesic agents, inhibiting 95%, 75%, 92% and 89%, respectively (FIG. 4—B, C, D and E).

The compounds obtained here are used as active principles for formulations reduce inflammatory processes and relieve pain, similar to what is reached by non-steroidal analgesic-anti-inflammatories. Some of them, such as (-)-hinokinin (6) and (-)-6,6′-diaminohinokinin (8), present similar power to indomethacin, but the gastric effects as observed for indomethacin are not evident for both prototypes. Therefore, the lowest side effects over the digestive system, added to the non-occurrence of other biochemical and hematological disturbances in preliminary tests demonstrate the advantage of these active principles over reference standard used. Thus, said substances may be used for diseases such as rheumatoid arthritis, tendonitis, periodontitis, bursitis and others.

### EXAMPLES

Tests with mice and rats were made and showed that the substances used are efficient to reduce inflammatory processes and pain, as we show in the figures below.

FIG. 3 shows graphs of the effect of oral administration of (-)-cubebin (1), (-)—O-benzyl cubebin (5), (-)-hinokinin (6), (-)-6,6′-dinitroinokinin (7) and (-)-6,6′-diamin (8) in doses of 10, 20, 30 and 40 mg/kg in the rat foot edema induced by carrageenin (100 μg/foot). Each bar represents the average ±SE (n=6) of the increase in edema volume (third hour) after the injection of carrageenin. Data were analyzed by one-way ANOVA and by Dunnett's multiple comparison variation test and the statistical significance was made for the level of p<0.05 (*) and p<0.01 (**).

FIG. 4 shows graphs of the effect of oral administration of (-)-cubebin (1), (-)—O-benzyl cubebin (5), (-)-hinokinin (6), (-)-6,6′-dinitroinokinin (7) and (-)-6,6′-diaminohiniokinin (8) in doses of 10, 20, 30 and 40 mg/kg for the writhing test induced by intraperitoneal injection of a 0.6% acetic acid solution in mice. Each bar represents the average ±SE (n=6) of the number of writhing in 20 minutes for different doses. Data were analyzed by one-way ANOVA and by Dunnett's multiple comparison variation test and the statistical significance was made for the level of p<0.05 (*) and p<0,01 (**)

1. Process to obtain dibenzylbutyrolactonic lignans, from (-)-cubebin (1) and from methylpluviatolide (9) of the structural formulas: 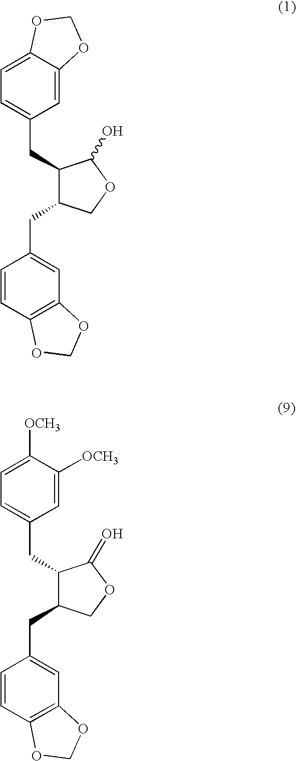
the process comprising:
a) collecting Zanthoxylum naranjillo leaves as a raw material and drying them in an oven at a temperature from 40 to 60° C.;
b) grinding Zanthoxylum naranjillo leaves into a ground powder
c) macerating the powder obtained from the Zanthoxylum naranjillo leaves and exhaustively extracting with hexane at 25° C. for about five days, to form a crude extract;
d) preparing the crude extract by filtering the maceration product and its concentration under reduced pressure a temperature of 30° C. until the complete elimination of the solvent;
e) repeatedly of purifying the crude extract obtained from step d) in a chromatographic column over silica gel and elution with solvent system starting with hexane, AcOEt (AcOEt) and ethanol in increasing proportions, supplying 210 chromatographic portions of 500 ml each;
f) obtaining and of isolating (-)-cubebin (1) and methylpluviatolide (9) from the chromatographic portions by crystallization (hexane/acetone (Me₂CO, 4:1) or thin layer preparative chromatography (hexane/Me₂CO, 4:1);
g) identification made identifying (-)-cubebin (1) and methylpluviatolide (9) by analysis of data obtained from nuclear magnetic resonance (NMR) of ¹H and ¹³C [α]_{D}, Mass, IV.

2. Process to obtain dibenzylbutyrolactonic lignans, from (-)-cubebin (1), the process comprising:
a) collecting Piper cubeba seeds as a raw material and drying them in an oven at a temperature between 40 and 60° C.;
b) grinding the Piper cubeba seeds into a ground powder;
c) macerating the ground powder obtained from the of Piper cubeba seeds and exhaustively extracting with 98% ethanol for 25° C. for five days to form a crude extract;
d) preparing the crude extract by filtering the maceration product and its concentration under reduced pressure at temperature of 40° C. until the complete elimination of the solvent;
e) solubilizing the crude ethanol extract in a 9:1 hydro alcohol solution of methanol and partition with n-hexane to eliminate the terpenoid oil portion;
f) separating the hydro alcohol portion and its later concentration until complete elimination of the solvents;
g) carrying out of vacuum liquid chromatography over silica gel of the crude hydro alcohol portion, using the following solvent systems: 100% hexane, 50% hexane: dichloromethane; 100% dichloromethane; 50% dichloromethane: ethyl acetate and 100% ethyl acetate;
h) carrying out vacuum elimination of the solvent from the portion in 100% dichloromethane and its successive recrystallizations in 4:1 hexane: acetone for (-)-cubebin (1) purification;
i) carrying out purity analysis of the crystallized (-)-cubebin (1) in thin layer chromatography and high performance liquid chromatography;
j) identifying (-)-cubebin (1) by analysis of data obtained from nuclear magnetic resonance (NMR) of ¹H and ¹³C [α]_{D}, Mass, IV.

3. Process to obtain tetrahydrofuranic lignans, from galgravin (12) and from veragensin (13), comprising:
a) collecting Nectandra megapotamica husks as a raw material and drying them in an oven at temperature between 40 and 60° C.;
b) milling the Nectandra megapotamica husks into a powder a knife
c) macerating the powder obtained from the Nectandra megapotamica husks and exhaustively extracting with EtOH: H₂O (9:1) at 25° C. for five days to form a crude extract;
d) preparing, the crude extract by filtering the maceration product and its concentration under reduced pressure at the temperature of 30° C. to complete the full elimination of the solvent;
e) fractioning of the crude extract by dissolution with MeOH: H₂O (7:3) and repeated partitions with hexane, chloroform and butanol, followed by lyophilization of the remaining water fraction, said fractions submitted to flash column chromatography over silica, using hexane-EtOAc (9:1) as mobile phase followed by semi-preparative HPLC (high performance liquid chromatography) (MeOH—H₂O 75: 250), obtaining the compounds galgravin (12) and veragensin (13);
f) made identifying galgravin (12) and veragensin (13) by analysis of data obtained from nuclear magnetic resonance (NMR) of ¹H and ¹³C [α]_{D}, Mass, IV.

4. Process to obtain synthetic and semi-synthetic derivatives from dibenzylbutyrolactonic lignans, derivatives of (-)-cubebin (1) and methylpluviatolide (9), as well as tetrahydrofuranic lignans, from galgravin (12) and from veragensin (13), comprising synthesis and semi-synthesis for dibenzylbutyrolactonic lignans and isolation from crude hydroalcoholic extract of N. megapotamica for tetrahydrofuranic lignans.

5. Process to obtain synthetic and semi-synthetic derivatives of dibenzylbutyrolatonic lignans, derivatives of (-)-cubebin (1) of claim 1, further comprising obtaining (-)—O-acetyl cubebin (2), (-)—O-methyl cubebin (3), (-)—O—(N,N-dimethylaminoethyl)-cubebin (4), (-)—O-benzyl cubebin (5), (-)-hinokinin (6), (-)-6,6′-dinitroinokinin (7), (-)-6,6′-diaminohinokinin (8).

6. Process to obtain synthetic and semi-synthetic dibenzylbutyrolactonic lignane derivatives, methylpluviatolide (9) derivatives, of claim 1, further comprising obtaining 6,6′-dinitromethylpluviatolide (10) and 6,6′-diaminomethylpluviatolide (11).

7. Compound derived from lignan obtained by of the process described in claim 1, wherein the compound acts as an anti-inflammatory agent.

8. Compound derived from lignan obtained by of the process described in claim 1, wherein the compound acts as an analgesic.

9. Topical or systemic formulations comprising, as active principle, from 60 to 80% of the compound derived from lignan obtained by the process described in claim 1.

10. Use of the compounds obtained by the process described in claim 1 in medicine and formulations to combat rheumatoid arthritis, tendonitis, periodontitis, and bursitis and others.

11. Therapeutic method by using the compounds described in claim 7 as active principles for formulations and medicines to reduce inflammatory processes and relieve pain,

12. Process to obtain synthetic and semi-synthetic derivatives of dibenzylbutyrolatonic lignans, derivatives of (-)-cubebin (1) of claim 2, further comprising obtaining (-)—O-acetyl cubebin (2), (-)—O-methyl cubebin (3), (-)—O—(N,N-dimethylaminoethyl)-cubebin (4), (-)—O-benzyl cubebin (5), (-)-hinokinin (6), (-)-6,6′-dinitroinokinin (7), (-)-6,6′-diaminohinokinin (8).

13. Process to obtain synthetic and semi-synthetic dibenzylbutyrolactonic lignane derivatives, methylpluviatolide (9) derivatives, of claim 4, further comprising obtaining 6,6′-dinitromethylpluviatolide (10) and 6,6′-diaminomethylpluviatolide (11).

14. Compound derived from lignan obtained by the process described in claim 2, wherein the compound acts as an anti-inflammatory agent.

15. Compound derived from lignan obtained by the process described in claim 2, wherein the compound acts as an analgesic.

16. Topical or systemic formulations comprising, as active principle, from 60 to 80% of the compound derived from lignan obtained by the process described claim 2.

17. Use of the compounds obtained by any of the processes described in claim 2 in medicine and formulations to combat diseases such as rheumatoid arthritis, tendonitis, periodontitis, and bursitis.

18. Therapeutic method using the compounds described in claim 8 as active principles for formulations and medicines to reduce inflammatory processes and relieve pain.

19. Process to obtain synthetic and semi-synthetic derivatives of dibenzylbutyrolatonic lignans, derivatives of (-)-cubebin (1) of claim 4, further comprising obtaining (-)—O-acetyl cubebin (2), (-)—O-methyl cubebin (3), (-)—O—(N,N-dimethylaminoethyl)-cubebin (4), (-)—O-benzyl cubebin (5), (-)-hinokinin (6), (-)-6,6′-dinitroinokinin (7), (-)-6,6′-diaminohinokinin (8).

20. Compound derived from lignan obtained by the process described in claim 3, wherein the compound acts as an anti-inflammatory agent.
